(19) United States
(12) Patent Application Publication
(10) Pub. No.: US 2024/0242808 A1
(43) Pub. Date: Jul. 18, 2024

(54) DRUG DISPENSER CAPABLE OF MEASURING NON-CONTACT BIOMETRIC INFORMATION

(71) Applicant: EVER INFORMATION TECHNOLOGY CO., LTD., Daejeon (KR)

(72) Inventor: Jae Du CHUN, Daejeon (KR)

(21) Appl. No.: 18/217,920

(22) Filed: Jul. 3, 2023

(30) Foreign Application Priority Data
Jan. 12, 2023 (KR) 10-2023-0004517

(51) Int. Cl. G16H 20/13; A61J 7/00; G06Q 30/0207; G06T 7/00; G06V 10/82; G06V 40/16

(52) U.S. Cl. CPC G16H 20/13; A61J 7/0076; G06Q 30/0207; G06T 7/0012; G06V 10/82; G06V 40/174; A61J 2200/30; A61J 2200/70; A61J 2205/60; G06T 2207/20084; G06T 2207/30088; G06T 2207/30201

(57) ABSTRACT

A drug dispenser capable of measuring non-contact biometric information includes a drug supplier that puts in drugs and discharges stored drugs; a vision sensor that takes an image of a person who takes drugs; a controller that recognizes and identifies a user on the basis of the image taken by the vision sensor and measures biometric information by analyzing the taken image of the user; and a communication unit that transmits the information measured by the controller to the outside. Since a user is identified and vital signs of the user are measured with the user in unconsciousness, there is an effect that it is possible to increase convenience for a user and more effectively manage the health state of a user.

### BACKGROUND

The present disclosure relates to a drug dispenser capable of measuring non-contact biometric information. In detail, the present disclosure relates to device that can recognize a user and measure vital signs of the user on the basis of an image, predict medication compliance by statistically managing the medication state of the user, and make it possible to provide the user with an incentive in accordance with the predicted medication compliance so that motivation for medication is possible.

A drug means a substance administered to an organism to treat, alleviate, or prevent disease, injury, or other bodily abnormality.

In general, even healthy people take medicine several times a year and people with certain diseases may need to take medicine periodically throughout their lives.

Since each of these drugs is composed of different chemical components, each drug has a different process of absorption and dissolution in the human body. Accordingly, in most cases, the medication-taking times are set in advance.

Therefore, it is important to take the prescribed medication at a fixed time at regular intervals according to the composition and purpose of the medicine and the duration of the medicinal effect for the correct administration effect.

That is, the doctor or pharmacist, who writes the prescription for the medicine, guides the medicine to be taken, the time to take the medicine, and the method of taking the medicine according to the life pattern of the general public, such as after waking up, before meals, after meals, and before going to bed. However, the medication guidance is explained verbally by the doctor or the pharmacist, and thereafter, it is inevitable to rely entirely on the user's memory and attention during the medication taking process. Therefore, if individual users do not pay attention, it is difficult to expect effective administration according to the medication schedule.

In particular, in the case of modern people who compete against time, it is not easy to always carry the medicine and take the prescribed medicine correctly at a fixed time due to busy daily life when going out.

Moreover, in the case of the children and the elderly, attention may be weaker than that of the general public, so that it is difficult to take the medicine regularly according to the medicine-taking schedule at the correct time, if there is no separate medicine-taking management. In addition, it is not possible to remember whether or not to take the medicine, so that the case of misuse of the medicine may occur frequently.

Accordingly, technologies for a medication dispenser for medication management, which allows a user to supply medication according to a predetermined time by inputting and discharging medication necessary for taking medication, have been proposed.

For example, in Korean Patent Registration No. 10-1953750, the present inventor has been proposed technology that informs the taker of the drug intake situation by including a communication unit that transmits the taker's medication taking situation and dangerous situation to the terminal of the taker and the family thereof, etc. and a speaker that tells the taker to take the medicine and information about the medicine by a voice.

In addition, Korean Patent Application Laid-Open No. 10-2016-0134950 has been proposed a technique for managing a medication schedule capable of recognizing a user through a camera, microphone, short-range communication module, etc., and detecting whether or not the user is taking the medicine by photographing the user's medication within a certain distance in front of the personal dispenser (200).

In addition, Korea Patent Registration No. 10-1656084 proposes a technology for remotely monitoring a patient's condition in real time by transferring bio-signal information to a patient management server such as a hospital or nursing home.

As described above, various medication dispenser technologies for managing the medication and health status of the taker, such as a technology for recognizing the user, a technology for confirming the user's intake, and a technology for measuring and providing biometric information of the user to the medical institution, have been suggested

However, most of the conventional technologies use a contact biometric sensor to recognize the user's biometric signal, and the user's artificial action is required. Accordingly, when the user does not touch the skin to the biometric sensor, there is a problem in that the user's biometric information is acquired.

In addition, since the prior art does not recognize the patient's medication status or changes in biometric information before and after taking the medication, there is a problem in that it is difficult to closely know the change in health status due to the medication.

In addition, the prior art does not provide enough motivation to induce patients to actively take medicine.

### PATENT LITERATURE

Patent Literature 1: Korean Patent Registration No. 10-1953750 (Feb. 25, 2019; Drug dispenser for medication management)
Patent Literature 2: Korean Patent Application Laid-Open No. 10-2016-0134950 (Nov. 24, 2016; Apparatus for controlling a personal dispenser and method thereof)
Patent Literature 3: Korean Patent Registration No. 10-1656084 (Sep. 2, 2016; Patient monitoring apparatus capable of automatically discharging medicine package)

### SUMMARY OF THE INVENTION

The present disclosure has been made in an effort to solve the problems described above, and an objective of the present disclosure is to provide healthcare by distinguishing a plurality of users in a non-contact manner and measuring vital signs of the users.

Another objective of the present disclosure is to predict medication compliance by statistically managing the medication state of a user and to make it possible to provide the user with an incentive in accordance with the predicted medication compliance so that motivation for medication is possible.

Another objective of the present disclosure is to make it possible to induce correct medication and prevent health deterioration due to incorrect medication by verifying whether the medicines that a user takes coincide with the prescription through a three-step confirmation process.

According to one aspect of the present invention so as to accomplish these objects, there is provided to a drug dispenser capable of measuring non-contact biometric information, the drug dispenser including: a drug supplier that puts in drugs and discharges stored drugs; a vision sensor that takes an image of a person who takes drugs; a controller that recognizes and identifies a user on the basis of the image taken by the vision sensor and measures biometric information by analyzing the taken image of the user; and a communication unit that transmits the information measured by the controller to the outside.

At this time, the communication unit receives prescription information of the user from the outside and the drug supplier discharges drugs on the basis of the prescription information.

In addition, the controller includes a user identification module that recognizes and identifies a user on the basis of an image taken by the vision sensor; a medication management module that confirms whether a user took drugs and manages a medication state of the user on the basis of the image taken by the vision sensor; a vital sign detection module that measures biometric information by analyzing pixel variation component data in a skin region of the user's face in the taken image and detects vital signs on the basis of the measured biometric information; a stress estimation module that classifies facial expressions of a user through a learning model learning a classifier for facial expressions through a neural network and estimates a stress index by calculating collection information of facial expressions classified in a reference time period; and a medication compliance prediction module that predicts and calculates medication compliance on the basis of any one item of information of a medication time, a number of times of medication, and variation of vital signs before and after medication.

In addition, the medication management module recognizes drug information through the prescription information and checks whether drugs coincide with the prescription information when the drugs are put in and discharged on the basis of drug images taken by the vision sensor.

In addition, the controller transmits the predicted medication compliance information to a management server and the management server performs incentive processing for the user on the basis of the medication compliance information.

In addition, the incentive processing includes at least one of a medical expense discount, a premium discount, and a medication management fee discount.

In addition, the vital sign detection module measures vital signs of a user by inputting movement component data of the face and pixel variation component data of a preset plurality of measurement spot regions of the skin region, as input data, into a learning model.

### BRIEF DESCRIPTION OF THE DRAWINGS

The above and other objects, features and advantages of the present invention will be more apparent from the following detailed description taken in conjunction with the accompanying drawings, in which:

FIG. 1 is a configuration diagram of the medication management system according to the present disclosure;

FIG. 2 is a block diagram showing the configuration of a drug dispenser that can measure non-contact biometric information;

FIG. 3 is a block diagram showing the detailed functions of the controller, and each function blocks are as follows; and

FIG. 4 shows landmarks, measurement spot regions, and a facial contour in a facial image.

### REFERENCE SIGNS LIST

1: Medical facility terminal
2: Management server
3: User terminal
4: Drug dispenser
10: Vision sensor
20: Operation unit
30: Power unit
40: Communication unit
50: Drug supplier
60: Storage
70: Controller
71: User identification module
72: Medication management module
73: Vital sign detection module
74: Stress estimation module
75: Medication compliance prediction module

### DETAILED DESCRIPTION OF THE PREFERRED EMBODIMENT

Since the description of the present invention is a mere embodiment for structural and functional description, it must not be interpreted that the scope of the present invention is limited by the embodiments described in the text. That is, since the embodiments can be variously changed and have various forms, it should be understood that the scope of the invention includes the equivalents for realizing the technical concept. Also, since the specific embodiments do not include all objects and effects presented by the present invention, the scope of the present invention is not limited by them.

Unless differently defined, all the terms used here including technical or scientific terms have the same meaning with what is generally understood by one who has common knowledge in the technical field that this invention belongs to. The terms such as those defined in the dictionary commonly used will be interpreted to have the meanings matching with the meanings in the context of the related technologies. Unless clearly defined in this application, they are not interpreted as ideal or excessively formal meanings.

Hereafter, the preferred embodiment according to the present disclosure are described in detail with reference to the accompanying drawings.

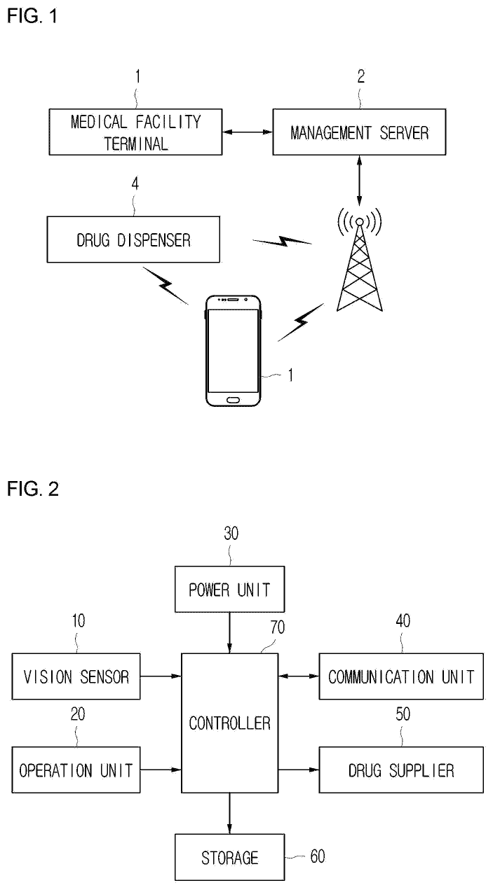

FIG. 1 is a configuration diagram of the medication management system according to the present disclosure.

Referring to FIG. 1, a medication management system includes a medical facility terminal (1), a management server (2), a user terminal (3), and a drug dispenser (4).

The medical facility terminal (1), which is a terminal that is installed at a medical facility such as a hospital and a pharmacy and can perform communication, performs a function of receiving and storing prescription information prescribed by medical staffs and transmitting the stored prescription information to the outside.

The management server (2) is a server disposed at a management company that manages medication of users. When receiving prescription information from the medical facility terminal (1), the management server (2) stores and manages the information and transmits prescription information to the user terminal (3) or the drug dispenser (4).

The user terminal (3), which is a terminal that is carried by a user who visits a medical facility and receives a prescription from the medical facility in relation to his/her disease, can receive prescription information from the management server (2) through the wired/wireless internet. An application for managing medication of the user is installed in the user terminal (3) so the user terminal (3) can have a function of monitoring and managing a medication state of the user.

The drug dispenser (4), which is an apparatus that receives drugs prescribed for a user and discharges drugs in accordance with the schedules set in prescriptions, can receive prescription information from the management server (2) through a wireless communication network.

There may be many methods for providing electronic prescription information. For example, there may be a method in which the medical facility terminal (1) transmits prescription information to an email account, an SNS account, etc. of a user and the drug dispenser (4) receives information stored in a cloud server, etc.

Further, it may be possible for the drug dispenser (4) to extract and store prescription information by photographing and scanning a prescription in a paper.

FIG. 2 is a block diagram showing the configuration of a drug dispenser that can measure non-contact biometric information.

As shown in FIG. 2, a drug dispenser (4) of the present disclosure includes a vision sensor (10), an operation unit (20), a power unit (30), a communication unit (40), a drug supplier (50), a storage (60), and a controller (70).

The vision sensor (10) serves to take images for recognizing a user and measuring vital signs. A camera may be used as the vision sensor (10).

The operation unit (20), which is for a user to perform functions such as information input and operation control, may include a touch display, an operation button, etc.

The power unit (30) supplies power for operating the drug dispenser (4), and a chargeable secondary battery may be used.

The communication unit (40) performs functions of receiving prescription information and medication schedule information and transmitting a medication state, vital signs, a stress index, medication compliance, etc. through communication with an external device.

The drug supplier (50) performs a function of discharging predetermined drugs in accordance with control by the controller (70).

The storage (60) stores prescription information and medication management information received from the outside and information such as vital signs, a stress index, and medication compliance created through the controller (70).

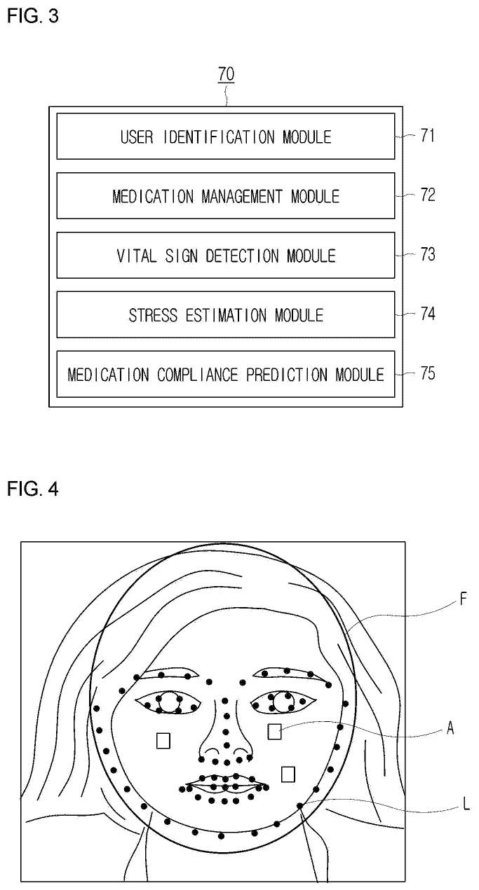

The controller (70) is a subject that controls the components and performs a medication management function for a user, and the detailed function that are performed by the controller (70) are described with reference to FIG. 3.

FIG. 3 is a block diagram showing the detailed functions of the controller, and each function blocks are as follows.

A user identification module (71), which performs a function of identifying a user on the basis of an image taken by the vision sensor (10), detects a facial region in a taken image, extracts a plurality feature points in the facial region, and checks whether the user is the same as a registered user by comparing the feature points with meta data stored in a library format. A technology of finding the centers of eyes and a mouth, performing rotation correction for vertical setting, and adjusting the pixel distances, etc. of the eyes and mouth may be applied to increase the face identification processing speed, and it is preferable to apply a jawline Region of Interest (ROI) technology and process an image after vectorization in order to increase the reliability in recognition.

In the present disclosure, the drug dispenser (4) may be shared by many users. To this end, prescription information or medication schedule information may be set for each user and facial feature information of a plurality of users may be stored.

To this end, it is possible to increase a recognition ratio and estimation accuracy by using neural network learning for facial images of users. In this case, a technology of coupling an encoder to a Single Shot MultiBox Detector (SSD) that can obtain feature maps of various scales may be applied to increase the facial recognition processing speed.

A medication management module (72) performs a function of controlling discharge of drugs on the basis of prescription information or medication schedule information set by a user and of confirming whether the user correctly takes drugs in accordance with the set schedule. Confirming whether a user takes drugs may be provided in the type of confirming whether the user took drugs and managing the medication state of the user on the basis of the image taken by the vision sensor, and it may also be possible to have a sound field sensor and recognize a sound that is generated when taking a drug.

In more detail, the method of confirming whether a user took drugs in the medication management module (72) may be composed of three steps.

As a first step, the medication management module (72) checks information such as the kind, amount, shape, color, and medication time of drugs to take by checking prescription information. The shape and color of drugs may be obtained from picture or image information of the drugs included in a prescription or by accessing big data about drugs that are provided by pharmaceutical institutions such as a pharmacy information center.

As a second step, the medication management module (72) checks whether the drugs that are taken coincide with prescription information on the basis of the prescription information. This process can be achieved by photographing drugs to be taken using the vision sensor (10) before putting drugs into the dispenser and then analyzing whether the taken drug images are the same as the images of the drugs included in the prescription information.

As a third step, the medication management module (72) confirms whether the drugs that are discharged for medication coincide with prescription information on the basis of the prescription information. This process can be achieved by photographing drugs that are discharged using the vision sensor (10) when the drugs are being discharged from the dispenser and then analyzing whether the taken drug images are the same as the images of the drugs included in the prescription information.

A vital sign detection module (73) obtains information such as the heart rate, body temperature, blood pressure, oxygen saturation, etc. of a user on the basis of an image taken by the vision sensor (10).

A heart rate can be obtained by, as a method of recognizing micro blood flow from image information and then estimating a heart rate from the micro blood flow, by comparing a color model of photoplethysmography (PPG) and the color of blood vessels in a facial region, inputting the comparison result into stochastic modeling of PPG, and then estimating a PPG value.

A blood pressure can be obtained through a method of creating a pulse wave signal on the basis of variation of brightness values of two different target regions in a skin region, estimating a pulse wave transmission time in the pulse wave signal, and estimating a blood pressure using the estimated pulse wave transmission time.

As for oxygen saturation, a region of interest is extracted from an image of a portion of a body such as a face, feature parameters are created on the basis of a color value of the region of interest, and then the oxygen saturation can be estimated on the basis of the average and the standard deviation of the feature parameters.

A stress estimation module (74) trains a classifier using a neural network for various facial expressions (anger, contempt, disgust, fear, happiness, absence of expression, sadness, surprise, etc.) corresponding to emotion in facial images of a user that are obtained in time series, and estimates a stress index on the basis of information of the numbers of occurrence of facial expressions classified by the neural network in a reference time period.

It is possible to increase a recognition ratio and estimation accuracy through neural network learning also when detecting vital signs. There is a problem that measurement accuracy is decreased by noise components due to movement of the face of a user when measuring vital signs in a facial region, and the same problem may occur even in a method through learning. Accordingly, it is preferable that the vital sign detection module (73) detects vital signs of a user by detecting a facial region in an image taken by the vision sensor (10) and then inputting facial movement component data and pixel variation component data of a plurality of measurement spot regions set in advance in a skin region into learning models, respectively.

In this case, it is preferable that the measurement spot region is a spot region of which similarity to biometric information of a user measured using pixel variation component data is higher than a reference value when compared to biometric information of a user measured using a contact type of biometric sensor, which will be described in detail with reference to FIG. 4.

A medication compliance prediction module (75) performs a function of predicting and calculating medication compliance on the basis of any one item of information of a medication time, a number of times of medication, and variation of vital signs before and after medication. In particular, when vital signs before and after medication is continuously improved as medication is performed in comparing the vital signs before and after medication, a high weight value may be applied when medication compliance is calculated.

The controller (70) transmits the predicted medication compliance information to the management server (2), and the management server (2) may be implemented to perform incentive processing for a user on the basis of the medication compliance of the user. Incentive processing may include at least any one of a medical expense discount, a premium discount, and a medication management fee discount. To this end, the subject that operates the management server (2) can make an affiliate business agreement for such services through discussion with a medical institution, an insurance company, etc.

FIG. 4 shows landmarks, measurement spot regions, and a facial contour in a facial image.

A plurality of landmarks (L) is shown as feature points in FIG. 4. Further, a facial contour (F) is a figure showing an approximate outline shape of a face formed in an elliptical shape to include the extracted landmarks (L).

In the present disclosure, the facial contour (F) can be used to estimate the distance between a user's face and the vision sensor (10) in accordance with the size of a figure and to correct color variation of pixels in accordance with the estimated distance or learn the degree of variation of measured vital signs.

Meanwhile, the measurement spot regions (A) are specific regions selected from a skin region to analyze pixel variation component data, and pixel variation component data are analyzed only for the measurement spot regions (A) rather than the entire skin region so that the measurement time can be reduced and the accuracy in measurement of biometric information can be enhanced.

The measurement spot regions (A) may be determined by selecting spot regions of which the similarity to biometric information of a user measured using pixel variation component data is higher than a reference value in comparison to biometric information of the user measured using a contact type of biometric sensor for a preset several spot regions.

Accordingly, an operation unit (20) measures biometric information by analyzing pixel variation component data for a plurality of preset measurement spot regions (A) and using the average of measurement values or the average of similar measurement values, etc.

According to the present disclosure, since a user is identified and vital signs of the user are measured with the user in unconsciousness, there is an effect that it is possible to increase convenience for a user and more effectively manage the health state of a user.

Further, since incentives are provided to users in accordance with medication compliance, users more actively take drugs, so there is an effect that it is possible to increase the health of users and anticipate early recovery.

While the present invention has been described with respect to the specific embodiments, it will be apparent to those skilled in the art that various changes and modifications may be made without departing from the spirit and scope of the invention as defined in the following claims.

What is claimed is:

1. A drug dispenser capable of measuring non-contact biometric information, the drug dispenser comprising:
a drug supplier that puts in drugs and discharges stored drugs;
a vision sensor that takes an image of a person who takes drugs;
a controller that recognizes and identifies a user on the basis of the image taken by the vision sensor and measures biometric information by analyzing the taken image of the user; and
a communication unit that transmits the information measured by the controller to the outside.

2. The drug dispenser of claim 1, wherein the communication unit receives prescription information of the user from the outside and the drug supplier discharges drugs on the basis of the prescription information.

3. The drug dispenser of claim 2, wherein the controller includes:
a user identification module that recognizes and identifies a user on the basis of an image taken by the vision sensor;
a medication management module that confirms whether a user took drugs and manages a medication state of the user on the basis of the image taken by the vision sensor; and
a vital sign detection module that measures biometric information by analyzing pixel variation component data in a skin region of the user's face in the taken image and detects vital signs on the basis of the measured biometric information.

4. The drug dispenser of claim 3, wherein the medication management module recognizes drug information through the prescription information and checks whether drugs coincide with the prescription information when the drugs are put in and discharged on the basis of drug images taken by the vision sensor.

5. The drug dispenser of claim 3, wherein the controller further includes a stress estimation module that classifies facial expressions of a user through a learning model learning a classifier for facial expressions through a neural network and estimates a stress index by calculating collection information of facial expressions classified in a reference time period.

6. The drug dispenser of claim 3, wherein the controller further includes a medication compliance prediction module that predicts and calculates medication compliance on the basis of any one item of information of a medication time, a number of times of medication, and variation of vital signs before and after medication.

7. The drug dispenser of claim 6, wherein the controller transmits the predicted medication compliance information to a management server and the management server performs incentive processing for the user on the basis of the medication compliance information.

8. The drug dispenser of claim 7, wherein the incentive processing includes at least one of a medical expense discount, a premium discount, and a medication management fee discount.

9. The drug dispenser of claim 3, wherein the vital sign detection module measures vital signs of a user by inputting movement component data of the face and pixel variation component data of a preset plurality of measurement spot regions of the skin region, as input data, into a learning model.
